(19) United States
(12) Patent Application Publication
(10) Pub. No.: US 2019/0021661 A1
(43) Pub. Date: Jan. 24, 2019

(54) PORTABLE ELECTRONIC APPARATUS

(71) Applicant: SEIKO EPSON CORPORATION, Tokyo (JP)

(72) Inventors: Hironori HASEI, Azumino-shi (JP); Noriaki HIRAIDE, Shiojiri-shi (JP)

(73) Assignee: SEIKO EPSON CORPORATION, Tokyo (JP)

(21) Appl. No.: 16/022,781

(22) Filed: Jun. 29, 2018

(30) Foreign Application Priority Data
Jul. 19, 2017 (JP) 2017-139735
Feb. 15, 2018 (JP) 2018-024800

(51) Int. Cl. A61B 5/00; H02J 7/35; G06F 1/16; A61B 5/0205

(52) U.S. Cl. CPC A61B 5/681; H02J 7/35; G06F 1/163; G06F 1/1635; A61B 5/02416; A61B 5/7425; G06F 1/1637; G06F 1/1698; A61B 5/0205

(57) ABSTRACT

A wrist apparatus as a portable electronic apparatus includes a case section having a rear surface as a second surface brought into contact with a user's body, a solar battery unit and a display unit provided side by side on a first surface side located on an opposite side to the second surface, a band portion used to mount the case section on the user's body, and a circuit board disposed in the case section and electrically connected to the solar battery unit and the display unit.

This application claims the benefit of JP 2017-139735, filed Jul. 19, 2017 and JP 2018-024800, filed Feb. 15, 2018. The disclosure of the prior applications is hereby incorporated by reference herein in their entirety.

### BACKGROUND

### 1. Technical Field

The present invention relates to a portable electronic apparatus.

### 2. Related Art

In the related art, there is a portable electronic apparatus such as a wrist apparatus which is mounted on a user's body, and has a function of measuring biological information such as a pulse wave of the user. For example, JP-A-2006-320735 discloses a wearable life support apparatus which is mounted on the body of a wearer, and acquires biological information or body motion information by using a pulse wave sensor or an acceleration sensor. In the wearable life support apparatus, since various sensors are operated to acquire biological information or body motion information for a long period of time (for example, a week), and thus power consumption increases, a method for reducing power consumption is proposed through power source management, for example, message display to a user is turned off during sleeping, or a specific sensor is stopped during sleeping.

However, as described above, power consumption increases in an apparatus mounted with various sensors, and thus there is a problem in that it is hard to continuously perform measurement by using the sensors for a long period of time.

### SUMMARY

An advantage of some aspects of the invention is to solve at least a part of the problems described above, and the invention can be implemented as the following forms or application examples.

### Application Example 1

A portable electronic apparatus according to this application example includes a solar battery unit; a secondary battery that stores power from the solar battery unit; a display unit that displays information to a user; a circuit board that is electrically connected to the solar battery unit, the secondary battery, and the display unit; a body motion sensor that is provided on the circuit board and detects body motion of the user; a biological sensor that measures biological information of the user; and a case section that has a first surface on which the solar battery unit and the display unit are provided side by side, and a second surface brought into contact with the user.

The portable electronic apparatus according to this application example includes the solar battery unit, the secondary battery that stores power from the solar battery unit, the display unit that displays information to a user, the circuit board, the body motion sensor, the biological sensor, and the case section. The case section has a first surface on which the solar battery unit and the display unit are provided side by side, and a second surface brought into contact with the user. With this configuration, it is possible to implement the portable electronic apparatus which can perform measurement for a long period of time. The solar battery unit and the display unit are disposed side by side on the first surface, and thus thinning of the apparatus can be realized.

### Application Example 2

In the portable electronic apparatus according to the application example, it is preferable that the biological sensor does not overlap the body motion sensor in a plan view from a normal direction to a light reception surface of the solar battery unit.

According to this application example, since the biological sensor and the body motion sensor are disposed at positions not overlapping each other in the plan view, the case section can be thinned.

### Application Example 3

In the portable electronic apparatus according to the application example, it is preferable that the solar battery unit includes a solar panel, and a first protection member protecting the solar panel, the display unit includes a display panel, and a second protection member protecting the display panel, and the first protection member and the second protection member are disposed on the first surface.

According to this application example, the solar battery unit (solar panel) and the display unit (display panel) disposed side by side can be protected by the first protection member and the second protection member disposed on the first surface.

### Application Example 4

In the portable electronic apparatus according to the application example, it is preferable that the first protection member and the second protection member are the same member.

According to this application example, the first protection member and the second protection member can be provided in both of the solar battery unit and the display unit through arrangement work (for example, coating work) performed once.

### Application Example 5

In the portable electronic apparatus according to the application example, it is preferable that an area of the solar panel is larger than an area of the display panel in a plan view from a normal direction to a light reception surface of the solar battery unit.

According to this application example, since an area of the solar panel is larger than an area of the display panel in the plan view, a sufficient power generation amount in the solar battery unit for securing power in the portable electronic apparatus can be obtained.

### Application Example 6

It is preferable that the portable electronic apparatus according to the application example further includes an antenna that is electrically connected to the circuit board, and is able to receive a radio signal, and the antenna is disposed between the solar panel and the case section in a plan view from a normal direction to a light reception surface of the solar battery unit.

According to this application example, since the antenna is disposed between the solar panel and the case section in the plan view, that is, the antenna is disposed on an outer peripheral side of the case section, it is possible to increase a degree of freedom of a disposition layout of the display unit, the solar panel, or the like and thus to more effectively dispose the display unit, the solar panel, or the like.

### Application Example 7

In the portable electronic apparatus according to the application example, it is preferable that the biological sensor is provided on the second surface.

According to this application example, since the biological sensor is provided on the second surface, contact between the biological sensor and the user can be performed favorably, and thus it is possible to increase detection accuracy.

### Application Example 8

In the portable electronic apparatus according to the application example, it is preferable that the biological sensor includes a light emitting portion, a light receiving portion, and a light transmissive portion, and the second surface has an opening, and the light transmissive portion is disposed in the opening.

According to this application example, since the light transmissive portion is disposed in the opening of the second surface, the light transmissive portion can be reliably brought into contact with the user, so that it is possible to suppress external light from entering the light receiving portion, and thus the influence of the external light on the biological sensor can be reduced.

### Application Example 9

In the portable electronic apparatus according to the application example, it is preferable that the secondary battery is disposed between the circuit board and the second surface.

According to this application example, since the secondary battery is disposed at a position where the solar battery unit or the display unit is not disposed in the case section on the second surface side, a size of the secondary battery can be increased, and thus it is possible to secure a larger charging amount.

### Application Example 10

In the portable electronic apparatus according to the application example, it is preferable that the secondary battery is disposed between the circuit board and the biological sensor.

According to this application example, it is possible to block so-called stray light which is light incident toward the solar battery unit for power generation but enters the inside of the case main body as leakage light through gaps or the like from the solar battery unit side, with the secondary battery, and can thus to reduce the influence of external light on the biological sensor.

### Application Example 11

In the portable electronic apparatus according to the application example, it is preferable that the secondary battery is disposed between the solar battery unit and the biological sensor.

According to this application example, a distance between the solar battery unit and the biological sensor can be secured due to the disposition of the secondary battery, and thus it is possible to reduce the influence of so-called stray light which is light incident toward the solar battery unit for power generation but enters the inside of the case as leakage light through gaps or the like from the solar battery unit side, on the biological sensor.

### BRIEF DESCRIPTION OF THE DRAWINGS

The invention will be described with reference to the accompanying drawings, wherein like numbers reference like elements.

FIG. 1 is a schematic configuration diagram illustrating a summary of a workout support system to which a wrist apparatus as a portable electronic apparatus is applied.

FIG. 2 is an exterior perspective view from a front side (display surface side) illustrating a schematic configuration of the wrist apparatus.

FIG. 3 is an exterior perspective view from a rear side illustrating a schematic configuration of the wrist apparatus.

FIG. 4 is a sectional view illustrating a configuration of the wrist apparatus.

FIG. 5 is a plan view illustrating a configuration of the wrist apparatus.

FIG. 6 is a functional block diagram illustrating a schematic configuration of the wrist apparatus.

FIG. 7 is a sectional view illustrating Modification Example 1 related to a configuration of the wrist apparatus.

FIG. 8 is a sectional view illustrating Modification Example 2 related to a configuration of the wrist apparatus.

FIG. 9 is a plan view illustrating Modification Example 3 related to a configuration of the wrist apparatus.

FIG. 10 is a plan view illustrating Modification Example 4 related to a configuration of the wrist apparatus.

FIG. 11 is a sectional view illustrating Modification Example 4 related to a configuration of the wrist apparatus.

### DESCRIPTION OF EXEMPLARY EMBODIMENTS

Hereinafter, embodiments of a system related to the invention will be described. The embodiments described below are not intended to improperly limit the content of the invention disclosed in the appended claims. All constituent elements described in each embodiment are not essential constituent elements of the invention.

### 1. Method of Present Embodiment

First, a description will be made of a workout support system as an example of a system to which a portable electronic apparatus according to the present embodiment of the invention is applied. Hereinafter, as an example of a portable electronic apparatus, a description will be made of a wrist apparatus (wearable apparatus) which is mounted on the wrist of a user and includes a pulse wave sensor or a body motion sensor.

A wrist apparatus as a portable electronic apparatus used for a workout support system is provided with a solar battery on a display unit side, and includes a pulse wave sensor as a biological sensor acquiring pulse wave information as biological information of a user or a body motion sensor acquiring action information of the user. The wrist apparatus includes a global positioning system (GPS) acquiring position information of the user as an example of a positioning system using a position information satellite called a global navigation satellite system (GNSS). A portable electronic apparatus is not limited to the wrist apparatus, and may be a wearable apparatus which is mounted on other parts of the user, such as the neck or the ankle.

The pulse wave sensor as an example of a biological sensor acquires pulse wave information such as a pulse rate. As the pulse wave sensor, for example, a photoelectric sensor (optical sensor) is used. In this case, the photoelectric sensor may detect reflected light or transmitted light of light applied to a living body. Since an amount of applied light absorbed or reflected in the living body differs depending on a blood flow rate in a blood vessel, sensor information detected by the photoelectric sensor is converted into a signal corresponding to the blood flow rate, and information regarding pulsation can be acquired by analyzing the signal. However, a pulse wave sensor is not limited to a photoelectric sensor, and may employ other sensors such as an electrocardiograph or an ultrasonic sensor.

The photoelectric sensor (optical sensor) is required to receive necessary light and to block unnecessary light. For example, in a case of a pulse wave sensor, reflected light including a pulse wave component reflected at a subject (particularly, a part including a measurement target blood vessel) which is a measurement target object is required to be received, and other light is a noise component and is thus required to be blocked.

The body motion sensor is a sensor detecting motion of the user. As the body motion sensor, an acceleration sensor, an angular velocity sensor, an azimuth sensor (geomagnetic sensor), a pressure sensor (altitude sensor), or the like may be used, but other sensors may be used.

The GPS stands for a global positioning system, and is a satellite positioning system for measuring the current position on the earth on the basis of a plurality of satellite signals. The GPS has a function of acquiring position information of a user by performing positioning calculation by using GPS time information and orbit information, and a time correction function in a clock function.

### 2. Workout Support System

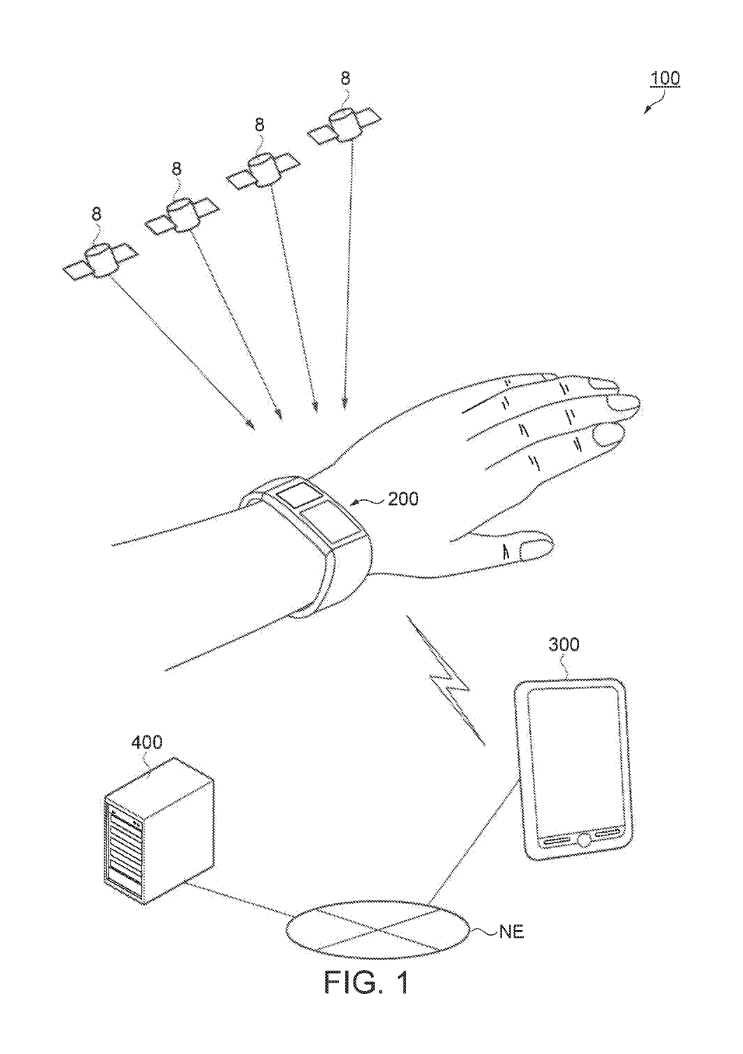

Next, with reference to FIG. 1, a description will be made of a configuration of a workout support system to which a wrist apparatus as a portable electronic apparatus is applied. FIG. 1 is a schematic configuration diagram illustrating a summary of a workout support system to which a wrist apparatus as a portable electronic apparatus is applied.

A workout support system 100 according to the present embodiment includes, as illustrated in FIG. 1, a wrist apparatus 200 as a portable electronic apparatus which is a detection apparatus including a pulse wave sensor as a biological sensor (photoelectric sensor) and a GPS; a portable apparatus 300 as a workout support apparatus; and a server 400 as an information processing apparatus which is connected to the portable apparatus 300 via a network NE.

The GPS as a global navigation satellite system provided in the wrist apparatus 200 has a function of receiving electric waves (satellite signals) from GPS satellites 8, and correcting internal time or acquiring position information by positioning calculation. Each of the GPS satellites 8 is an example of a position information satellite which orbits on a predetermined orbit in the sky above the earth and transmits high-frequency electric waves superimposed with a navigation message to the ground. In the following description, an electric wave superimposed with a navigation message will be referred to as a satellite signal.

A satellite signal from the GPS satellite 8 includes GPS time information which is considerably accurate, and a time correction parameter for correcting a time error. The wrist apparatus 200 may receive a satellite signal (electric wave) from a single GPS satellite 8 so as to acquire time information by using the GPS time information and the time correction parameter included therein.

The satellite signal also includes orbit information indicating a position on an orbit of the GPS satellite 8. The wrist apparatus 200 may perform positioning calculation by using the GPS time information and the orbit information. The positioning calculation is performed on the premise that some extent of an error is included in an internal time of the wrist apparatus 200. In other words, a time error is also an unknown number in addition to x, y and z parameters for specifying a three-dimensional position of the wrist apparatus 200. Thus, the wrist apparatus 200 may receive satellite signals (electric waves) transmitted from, for example, three or more GPS satellites 8, and may perform positioning calculation by using GPS time information and orbit information included therein so as to acquire position information of the current location.

The portable apparatus 300 as a workout support apparatus may be formed of, for example, a smart phone or a tablet terminal apparatus. The portable apparatus 300 is connected to the wrist apparatus 200 in which a pulse wave sensor is used as a biological sensor which is a photoelectric sensor via short-range radio communication such as Bluetooth (registered trademark) communication or wired communication (not illustrated). The portable apparatus 300 receives measurement information from the wrist apparatus 200, and notifies a user of processed pulse wave information or body motion information of the user, or position information. However, the portable apparatus 300 may be variously modified, for example, by including an optical sensor unit 40, a body motion sensor unit 170 as a body motion sensor, or a GPS reception unit 160 which will be described later included in the wrist apparatus 200.

The wrist apparatus 200 and the portable apparatus 300 have a Bluetooth function, and the portable apparatus 300 and the wrist apparatus 200 are connected to each other via Bluetooth communication, for example, Bluetooth Low Energy (also called Bluetooth 4.0). Bluetooth Low Energy focuses on power saving, and can considerably save power compared with an old version so as to increase available time of the wrist apparatus.

The portable apparatus 300 may be connected to the server 400 such as a personal computer (PC) or a server system via the network NE. The network NE here may employ various networks NE such as a wide area network (WAN), a local area network (LAN), a mobile phone communication network, and short-range radio communication. In this case, the server 400 is realized as a processing storage unit which receives pulse wave information or body motion information measured by the wrist apparatus 200 or data processed by the portable apparatus 300 from the portable apparatus 300 via the network NE, and stores the information or the data.

In the embodiment, the wrist apparatus 200 is not required to be directly connected to the network NE as long as the wrist apparatus 200 can perform communication with the portable apparatus 300. Therefore, a configuration of the wrist apparatus 200 can be simplified. However, in the workout support system 100, a modification may occur in which the portable apparatus 300 is omitted, and the wrist apparatus 200 is directly connected to the server 400. In this case, the wrist apparatus 200 has a function, which is a function of the portable apparatus 300, of processing measurement information, and a function of transmitting measurement information to the server 400 or receiving information from the server 400.

The workout support system 100 is not limited to a configuration including the server 400. For example, processes or functions performed in the workout support system 100 may be realized by the portable apparatus 300. For example, the portable apparatus 300 such as a smart phone has restrictions in processing capability, a storage region, and a battery capacity compared with a server system, but may secure sufficient processing capability and the like in consideration of the recent capability improvement. Therefore, if the needs for the processing capability and the like are satisfied, the portable apparatus 300 can independently realize processes or functions performed in the workout support system 100 according to the present embodiment.

The workout support system 100 according to the present embodiment is not limited to being realized by three apparatuses. For example, the workout support system 100 may include two or more apparatuses among the wrist apparatus 200, the portable apparatus 300, and the server 400. In this case, processes performed in the workout support system 100 may be performed by any one apparatus, and may be distributed to and performed by a plurality of apparatuses. The workout support system 100 according to the present embodiment may include apparatuses which are different from the wrist apparatus 200, the portable apparatus 300, and the server 400. However, in a case of taking into consideration improvement of terminal capability or a use form, there may be an embodiment in which the workout support system 100 according to the present embodiment is realized by the wrist apparatus 200.

The workout support system 100 of the present embodiment includes a memory storing information (for example, programs or pieces of data), and a processor which operates on the basis of the information stored in the memory. In the processor, for example, a function of each unit may be realized by individual hardware, and may be realized by integrated hardware. The processor may be, for example, a central processing unit (CPU). However, the processor is not limited to a CPU, and may employ various processors such as a graphics processing unit (GPU) or a digital signal processor (DSP). The processor may be a hardware circuit using an ASIC. The memory may be, for example, a semiconductor memory such as a static random access memory (SRAM) or a dynamic random access memory (DRAM), may be a register, may be a magnetic storage device such as a hard disk device, and may be an optical storage device such as an optical disc device. For example, the memory stores computer readable commands, and the commands are executed by the processor such that a function of each unit of the workout support system 100 is realized. The commands here may be commands forming a program, and may be commands for instructing a hardware circuit to perform an operation.

### 3. Wrist Apparatus

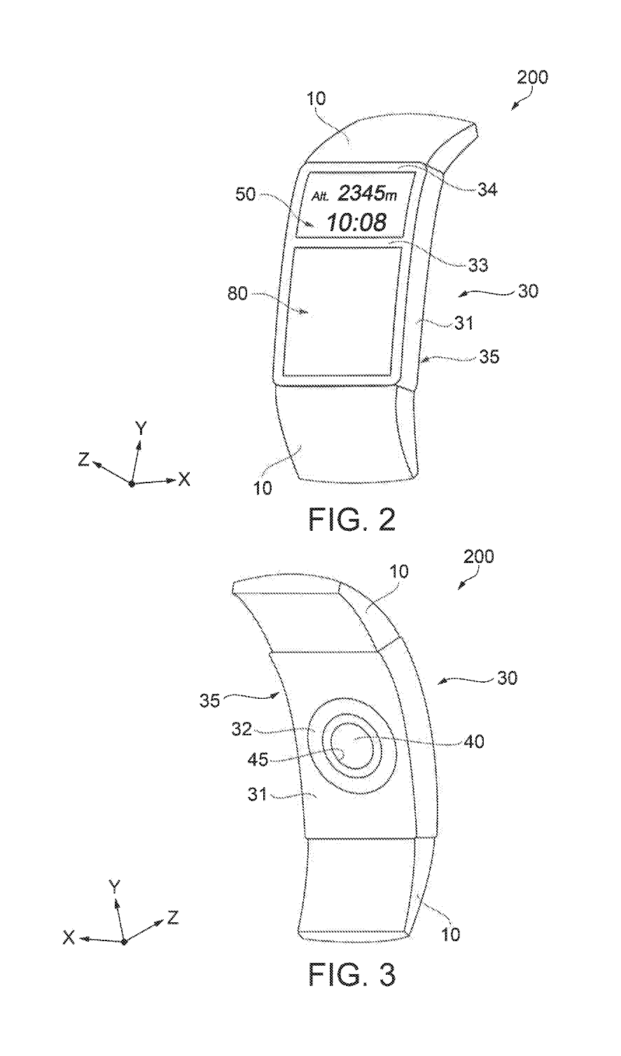

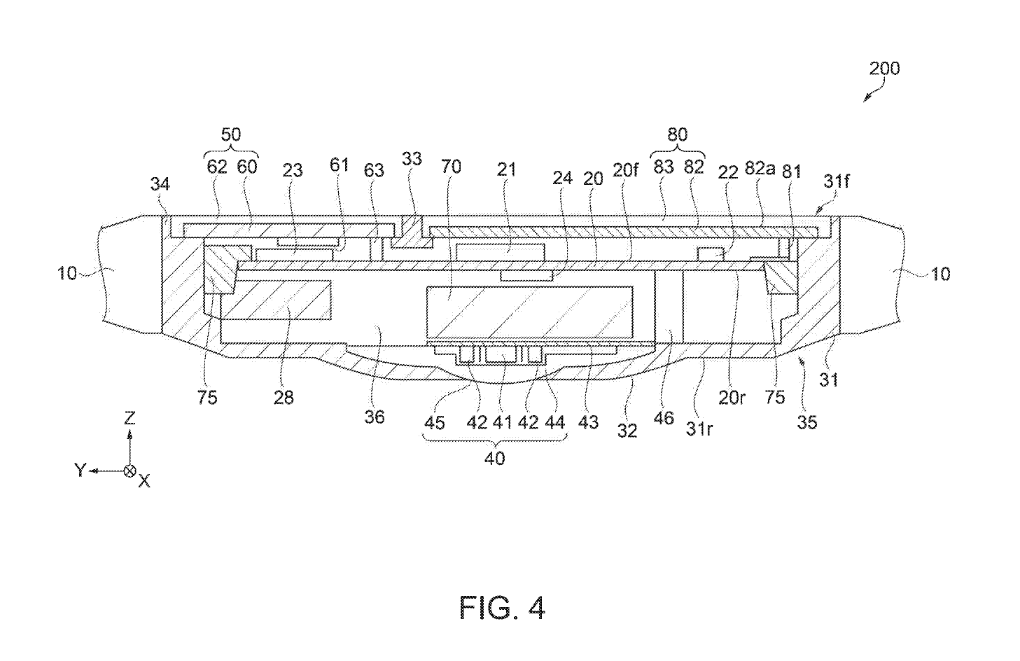

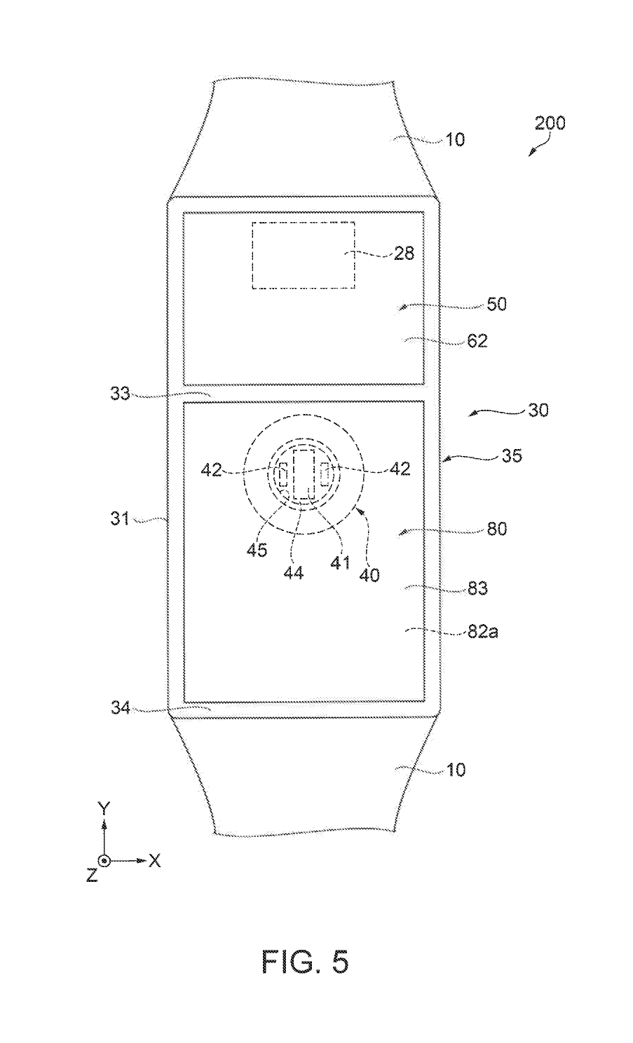

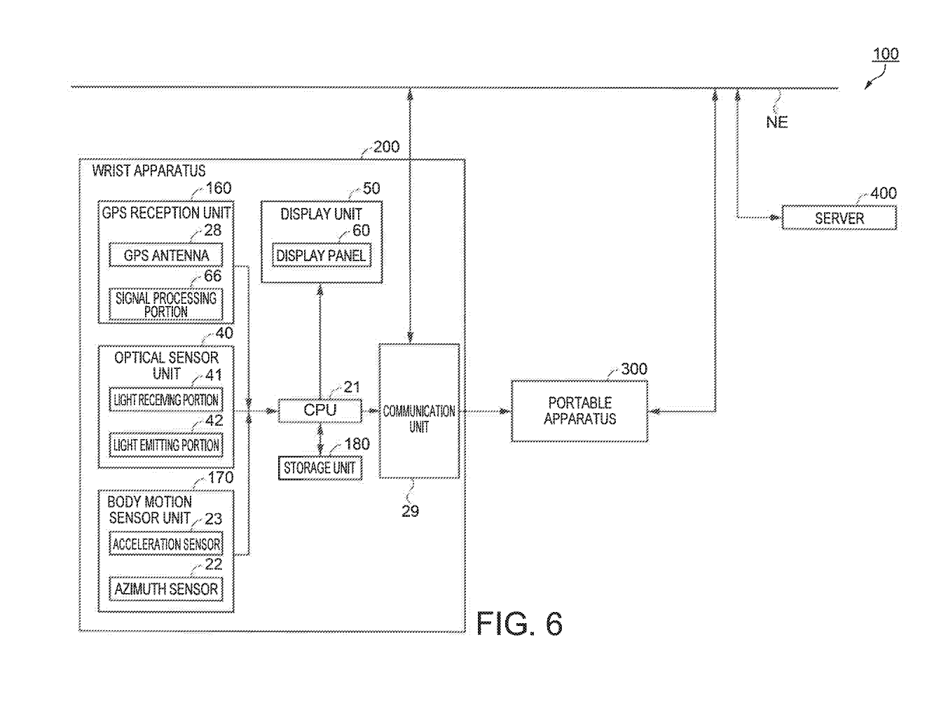

Next, with reference to FIGS. 2 to 6, a description will be made of a configuration of the wrist apparatus as a portable electronic apparatus. FIG. 2 is an exterior perspective view from a front side (display surface side) illustrating a schematic configuration of the wrist apparatus. FIG. 3 is an exterior perspective view from a rear side illustrating a schematic configuration of the wrist apparatus. FIG. 4 is a sectional view illustrating a configuration of the wrist apparatus. FIG. 5 is a plan view illustrating a configuration of the wrist apparatus. FIG. 6 is a functional block diagram illustrating a schematic configuration of the wrist apparatus.

In the following description of the wrist apparatus 200, when an apparatus main body 30 is worn by a user, a side located on a target object side which is a target part for measuring biological information or the like will be referred to as “a rear side or a rear surface side”, and a display surface side of the apparatus main body 30 opposite side thereto will be referred to as “a front side or a front surface side”. A measurement “target object (target part)” will be referred to as a “subject” in some cases. A coordinate system is set with a case main body 31 of the wrist apparatus 200 as a reference, and a direction which intersects the display surface of the display unit 50, and is directed from a rear surface toward a front surface corresponding to the display surface side of the display unit 50 is defined as a positive Z axis direction (+Z axis direction). Alternatively, a direction which is directed from the circuit board 20 toward the display unit 50, a direction which becomes distant from the case main body 31 in a normal direction to light reception surface 82a of a solar panel 82 forming a solar battery unit 80, or a direction which is directed from the circuit board 20 toward the solar battery unit 80 may be defined as a positive Z axis direction. In a state in which the wrist apparatus 200 is worn on a subject, the positive Z axis direction corresponds to a direction directed from the subject toward the case main body 31. Two axes orthogonal to the Z axis are defined as XY axes, and, particularly, a direction in which band portions 10 are attached to the case main body 31 or a direction directed from one end of the case main body 31 attached with the band portion 10 toward the other end thereof is set to the Y axis.

An axis orthogonal to the Z axis and the Y axis is set to the X axis. The light reception surface 82a is a surface via which light is incident to the solar battery unit 80.

FIG. 2 is a perspective view in which the wrist apparatus 200 to which the band portion 10 is fixed is viewed from the +Z axis direction which is a direction directed toward the front side (display unit 50 side) from the rear side corresponding to a subject side in a mounting state. FIG. 3 is a perspective view in which the wrist apparatus 200 is viewed from the rear side opposite to FIG. 2, that is, from the −Z axis direction. FIG. 4 is a sectional view in which the wrist apparatus 200 is viewed from the +Y axis direction.

As illustrated in FIGS. 2 to 4, the wrist apparatus 200 as a portable electronic apparatus is mounted on a predetermined part (for example, a measurement target part such as the wrist) of the user, and measures pulse wave information, position information, or the like. The wrist apparatus 200 includes the apparatus main body 30 which includes the case main body 31 and is in close contact with the user so as to measure pulse wave information or the like, and a pair of band portions 10 which is attached to the apparatus main body 30 and is used to mount the apparatus main body 30 on the user.

The apparatus main body 30 including the case main body 31 is provided with the display unit 50, the solar battery unit 80 including the solar panel 82 disposed side by side with the display unit 50, and a measurement window portion 45 corresponding to the optical sensor unit 40 as a biological sensor. An outer surface of the apparatus main body 30 may be provided with an operation unit (operation buttons) (not illustrated) or a display element such as an LED which delivers information through blinking or the like. However, the wrist apparatus 200 is not limited to such a configuration, and may be variously modified by omitting some of the constituent elements or adding other constituent elements thereto.

The apparatus main body 30 has the case main body 31 which is open on a front surface 31f side as a first surface. The case main body 31 which is open on the front surface 31f side has outer frame portions 34 provided along an outer periphery of the opening, and an intermediate frame portion 33 which connects the opposing outer frame portions 34 to each other, and thus divides an opening region formed by the outer frame portions 34 into two regions. The outer frame portions 34 and the intermediate frame portion 33 are provided with step parts at inner peripheries on the two separate regions sides. The display unit 50 is disposed in one region obtained through division using the outer frame portions 34 and the intermediate frame portion 33, and is inserted into the step parts of the outer frame portions 34 and the intermediate frame portion 33 so as to be attached. The solar battery unit 80 is disposed in the other region obtained through division using the outer frame portions 34 and the intermediate frame portion 33, and is inserted into the step parts of the outer frame portions 34 and the intermediate frame portion 33 so as to be attached. In other words, the display unit 50 and the solar battery unit 80 are provided side by side on the front surface 31f side of the case main body 31. An internal space 36 which is a closed space is provided in the inside of the case main body 31 surrounded by the case main body 31, and the display unit 50 and the solar battery unit 80 closing the opening of the case main body 31.

The display unit 50 includes a liquid crystal display (hereinafter, referred to as a display panel 60) attached to the front surface 31f side as a first surface of the outer frame portions 34 and the intermediate frame portion 33 in one region, and a second protection member 62 which covers the display panel 60 from the front side (+Z axis direction side) and is provided to protect the display panel 60 from the front side. In the display unit 50 with this configuration, the second protection member 62 is exposed to the front surface 31f side as a first surface located on an opposite side to a rear surface 31r as a second surface which is a rear side of the case main body 31 and is a contact surface with the user's body. In other words, in the display unit 50 located on the front surface side of the apparatus main body 30, the second protection member 62 is exposed to the front side. Therefore, the display unit 50 (display panel 60) located on the front surface side can be protected by the second protection member 62. The second protection member 62 may employ a light-transmissive resin coating material or glass coating material. The second protection member 62 may cover surfaces including the front surface to the rear surface of the display panel 60. The display unit 50 may include an illumination unit 61 which irradiates the display panel 60 with light. As a modification example of the display unit 50, the display unit 50 may have a display configuration in which one or more display elements such as LEDs delivering information through blinking or the like, and a protection member covering the display configuration, and the protection member may be exposed to the front surface 31f side which is an opposite side to the rear surface 31r as a second surface.

The solar battery unit 80 includes the solar panel 82 attached to the outer frame portions 34 and the intermediate frame portion 33 in the other region, and a first protection member 83 which covers the light reception surface 82a of the solar panel 82 from the front side (+Z axis direction side) and is provided to protect the solar panel 82 from the front side. In the solar battery unit 80 with this configuration, the first protection member 83 is exposed to the front surface 31f as a first surface located on an opposite side to the rear surface 31r as a second surface which is a contact surface of the case main body 31 with the user. In other words, in the solar battery unit 80 located on the front surface side of the apparatus main body 30, the first protection member 83 is exposed to the front side. Therefore, the solar battery unit 80 (solar panel 82) located on the front surface side can be protected by the first protection member 83. The first protection member 83 may employ a light-transmissive resin coating material or glass coating material. The first protection member 83 may cover surfaces including the light reception surface 82a to the side surface of the solar panel 82.

Here, preferably, the first protection member 83 and the second protection member 62 are made of a light-transmissive resin coating material or glass coating material, and are formed of the same member. In a case where the first protection member 83 and the second protection member 62 are formed of the same member, the first protection member 83 and the second protection member 62 can be provided in both of the solar battery unit 80 and the display unit 50 through arrangement work (for example, coating work) performed once.

An area of the solar panel 82 (an area of the light reception surface 82a) forming the solar battery unit 80 is preferably larger than an area of the display panel 60 in a plan view along a direction from the display unit 50 toward the circuit board 20 (a plan view from the +Z axis direction). As mentioned above, since an area of the solar panel 82 (an area of the light reception surface 82a) is larger than an area of the display panel 60, a sufficient power generation amount in the solar battery unit 80 for securing power in the wrist apparatus 200 can be obtained.

The rear surface 31r which is a rear side surface of the case main body 31 and is a contact surface with the user's body, a protrusion portion 32 protruding from the rear surface 31r, and the measurement window portion 45 of the optical sensor unit 40 which is open to the top of the protrusion portion 32 is provided on the rear side of the case main body 31. The optical sensor unit 40 as a biological sensor is disposed at a position corresponding to the measurement window portion 45 in a plan view from the +Z axis direction, and a light transmissive portion 44 forming the optical sensor unit 40 is inserted into the measurement window portion 45.

The case main body 31 may be formed by using, for example, metal such as stainless steel, or a resin. A configuration of the case main body 31 is not limited to an integrated configuration, and may be a configuration of being divided into a plurality of parts, for example, the case main body 31 may have a dual structure in which a lid is provided on the side mounted on a user.

As illustrated in FIG. 4, as element components forming the wrist apparatus 200, for example, a circuit board 20, an azimuth sensor 22 and an acceleration sensor 23 as sensors included in the body motion sensor unit 170 (refer to FIG. 6), a GPS antenna 28 which can receive a radio signal, the optical sensor unit 40, the illumination unit 61 of the display panel 60, and a secondary battery 70 (lithium secondary battery) are stored in the internal space 36 of the case main body 31. However, the apparatus main body 30 is not limited to the configuration illustrated in FIG. 4, and may be added with other sensors such as a pressure sensor for calculating an elevation or a temperature sensor for measuring a temperature, or a vibrator. The circuit board 20 is connected to connection wires with the above-described element components, a central processing unit (CPU) 21 which is a control circuit controlling the respective sensors or the display unit 50 or a control circuit including a drive circuit, and other circuit elements 24.

Among the element components forming the wrist apparatus 200 disposed in the internal space 36, the display unit 50 and the solar battery unit 80 are disposed side by side on the opening side (front surface side), and the circuit board 20, the secondary battery 70, and the optical sensor unit 40 are disposed in this order on the rear surface side.

In other words, the secondary battery 70 is disposed between the circuit board 20 and the rear surface 31r which is a rear side surface of the case main body 31 and is a contact surface with the user's body. With this disposition, since the secondary battery 70 is disposed at a position where the solar battery unit 80 or the display unit 50 is not disposed in the case main body 31, a size of the secondary battery 70 can be increased, and thus it is possible to secure a larger charging amount.

The secondary battery 70 is preferably disposed between the circuit board 20 and the optical sensor unit 40. If the secondary battery 70 is disposed as mentioned above, it is possible to block so-called stray light which is light incident toward the solar battery unit 80 (the light reception surface 82a of the solar panel 82) for power generation but enters the inside of the case main body 31 as leakage light through various gaps or the like from the solar battery unit 80 side, with the secondary battery 70, and can thus to reduce the influence of external light (stray light) on the optical sensor unit 40.

The secondary battery 70 is preferably disposed between the solar battery unit 80 and the optical sensor unit 40. If the secondary battery 70 is disposed as mentioned above, a distance between the solar battery unit 80 and the optical sensor unit 40 can be secured, and thus it is possible to reduce the influence of so-called stray light which is light incident toward the solar battery unit 80 for power generation but enters the inside of the case as leakage light through gaps or the like from the solar battery unit 80 side, on the optical sensor unit 40.

Since the circuit board 20 is disposed between the solar battery unit 80 and the optical sensor unit 40 in the case main body 31, it is possible to block so-called stray light which is light incident toward the solar battery unit 80 for power generation but enters the inside of the case main body 31 as leakage light through gaps or the like from the solar battery unit 80 side, with the circuit board 20, and can thus to reduce the influence of external light (stray light) on the optical sensor unit 40.

Hereinafter, each element component will be described also with reference to the functional block diagram of FIG. 6.

The circuit board 20 has a front surface 20f and a rear surface 20r which is different from the front surface 20f and is an opposite surface to the front surface 20f, and ends thereof are attached to the inside of the case main body 31 via a circuit case 75. The azimuth sensor 22 and the acceleration sensor 23 as sensors included in the body motion sensor unit 170, the CPU 21 as a control circuit, and the like are mounted on the front surface 20f of the circuit board 20, and other circuit elements 24 and the like are mounted on the rear surface 20r.

In a plan view from the +Z axis direction, the optical sensor unit 40 as a biological sensor and the azimuth sensor 22 or the acceleration sensor 23 included in the body motion sensor unit 170 are preferably disposed not to overlap each other. As mentioned above, in a plan view from the +Z axis direction, since the optical sensor unit 40 and the azimuth sensor 22 or the acceleration sensor 23 included in the body motion sensor unit 170 do not overlap each other, a case section 35 can be thinned.

The display panel 60 and the solar battery unit 80 are connected to the front surface 20f of the circuit board 20 via a connection wiring portion 63 and a connection wiring portion 81 formed of flexible boards or the like. The optical sensor unit 40 is electrically connected to the rear surface 20r of the circuit board 20 which is an opposite surface to the front surface 20f via a connection wiring portion 46 formed of a flexible board or the like. With such disposition, routing of a wiring for connection can be made the minimum, and it is also possible to block stray light which is incident light for power generation but enters the inside of the case as leakage light through a gap or the like from the solar battery unit 80 side, with the circuit board 20, and can thus to reduce the influence of external light (stray light) on the optical sensor unit 40. The circuit case 75 can guide the secondary battery 70 or the like.

The azimuth sensor (geomagnetic sensor) 22 or the acceleration sensor 23 included in the body motion sensor unit 170 as a body motion sensor may measure information related to motion of the user's body, that is, body motion information. The azimuth sensor (geomagnetic sensor) 22 or the acceleration sensor 23 outputs a body motion detection signal which is a signal changing depending on movement or turning of the user, and transmits the body motion detection signal to the CPU 21 as a processing unit including a control circuit.

The CPU 21 forms a control circuit or the like controlling a circuit which controls the GPS reception unit 160 including the GPS antenna 28, a circuit which drives the optical sensor unit 40 so as to measure a pulse wave, a circuit which drives the display unit 50 (display panel 60), a circuit which drives the body motion sensor unit 170 so as to measure body motion information, and a power generation circuit in the solar battery unit 80. The CPU 21 transmits pulse wave information or body motion information measured at each part, or position information of the user to a communication unit 29 as necessary.

The GPS antenna 28 as an antenna which can receive a radio signal is included in the GPS reception unit 160 along with a signal processing portion 66, and receives a plurality of satellite signals. The signal processing portion 66 performs positioning calculation on the basis of the plurality of satellite signals received by the GPS antenna 28, and acquires position information of the user.

The GPS antenna 28 is electrically connected to the circuit board 20, and is preferably disposed as illustrated in FIG. 5 such that the solar battery unit 80 is disposed outside an outer edge of the GPS antenna 28 in a plan view from the +Z axis direction. As mentioned above, since the solar battery unit 80 is disposed outside the outer edge of the GPS antenna 28 in a plan view from the +Z axis direction, that is, the solar battery unit 80 and the GPS antenna 28 are disposed at positions not overlapping each other in a plan view, a satellite signal can reach the GPS antenna 28 in a favorable state without being blocked by the solar battery unit 80, and thus it is possible to increase reception sensitivity. Although not illustrated, in the same manner as the GPS antenna 28, an antenna which is included in the communication unit 29 and can receive a radio signal is preferably disposed at a position not overlapping the solar battery unit 80 in a plan view from the +Z axis direction, and, with this disposition, it is possible to favorably receive a radio signal.

The GPS antenna 28 is electrically connected to the circuit board 20, and is preferably disposed between the solar panel 82 and the case section (case main body 31) in a plan view from the +Z axis direction. As mentioned above, since, in a plan view from the +Z axis direction, the GPS antenna 28 is disposed between the solar battery panel 82 and the case section (case main body 31), that is, the GPS antenna 28 is disposed on the outer peripheral side of the case section (case main body 31), it is possible to increase a degree of freedom of a disposition layout of the display unit 50, the solar panel 82, or the like and thus to more effectively dispose the display unit 50, the solar panel 82, or the like.

The communication unit 29 transmits pulse wave information or body motion information, or position information of the user transmitted from the CPU 21 to the portable apparatus 300 or the like as necessary, or receives information from the portable apparatus 300.

The optical sensor unit 40 as a biological sensor measures a pulse wave or the like, and includes the light receiving portion 41, and a plurality of (in the present embodiment, two) light emitting portions 42 disposed on both sides of the light receiving portion 41, that is, outside of the light receiving portion 41 (on the outer peripheral side of the case main body 31) in a plan view. As mentioned above, since the light receiving portion 41 is disposed inside the light emitting portion 42, it is possible to prevent external light from the outer peripheral side of the case main body 31 from entering the light receiving portion 41, and thus to reduce the influence of the external light on the optical sensor unit 40. The number of light emitting portions 42 is not limited to two, and may be one or three or more. The light receiving portion 41 and the two light emitting portions 42 are attached to one surface of a sensor substrate 43, and is covered with the light transmissive portion 44 which is formed of a light-transmissive member made of a thermosetting resin. A portion of the light transmissive portion 44 including a region corresponding to the light receiving portion 41 and the two light emitting portions 42 is inserted into the measurement window portion 45 as an opening provided in the rear surface 31r of the case main body 31.

The light transmissive portion 44 may protrude from the top of the protrusion portion 32 of the case main body 31. In other words, the light transmissive portion 44 may be exposed from the rear surface 31r as a contact surface of the case section 35. As mentioned above, since the light transmissive portion 44 is exposed from the rear surface 31r as a contact surface, that is, the optical sensor unit 40 is exposed from the rear surface 31r, the light transmissive portion 44 can be reliably brought into contact with the user, so that it is possible to suppress external light from entering the light receiving portion 41, and thus the influence of the external light on the optical sensor unit 40 can be reduced.

As described above, in the optical sensor unit 40, a subject (measurement target object) is irradiated with light emitted from the light emitting portion 42, and reflected light is received by the light receiving portion 41, and thus pulse wave information is measured. The optical sensor unit 40 outputs a signal detected by the pulse wave sensor including the light emitting portion 42 and the light receiving portion 41, as a pulse wave measurement signal. For example, a photoelectric sensor is used as the optical sensor unit 40. In this case, there may be a method in which reflected light or transmitted light of light applied to a living body (the wrist of the user) from the light emitting portion 42 is detected by the light receiving portion 41. In this method, since an amount of applied light absorbed or reflected in the living body differs depending on a blood flow rate in a blood vessel, sensor information detected by the photoelectric sensor is converted into a signal corresponding to the blood flow rate, and information regarding pulsation can be acquired by analyzing the signal. However, a pulse wave sensor is not limited to a photoelectric sensor, and may employ other sensors such as an electrocardiograph or an ultrasonic sensor.

As illustrated in FIG. 5, the optical sensor unit 40 is disposed at the center of the apparatus main body 30 (case main body 31) in a plan view from the +Z axis direction. In other words, preferably, in a plan view from the +Z axis direction, the optical sensor unit 40 is disposed at a position overlapping the centroid of the case section (case main body 31), and overlaps at least a part of the solar battery unit 80. The centroid of the case section (case main body 31) may be replaced with the center of mass. In a case of a solid object, the centroid may be defined in a structure of the solid object, or may be defined in a space. The term “overlapping the centroid” may be defined as a state of overlapping the centroid in a case where the position of the centroid is projected onto a two-dimensional plane or a predetermined target object when viewed from a predetermined direction. Consequently, it is possible to suppress the influence of external light (leakage light) on the optical sensor unit 40 or to improve disposition balance of the apparatus main body 30 while increasing a sunlight power generation amount in the solar battery unit 80 as large as possible, and thus to facilitate detection in the optical sensor unit 40 and also to improve mountability of the apparatus main body 30 on the user.

The display unit 50 is configured to allow the user to visually recognize numbers, icons, or time display indicators displayed on a display member such as the display panel 60 through the second protection member 62. In other words, in the present embodiment, various pieces of information such as measured biological information or information indicating a workout state are displayed by using the display panel 60, and the display is presented to the user from the front side (in the +Z axis direction). As the display member, instead of the display panel 60 which is a liquid crystal display, an organic electroluminescence (EL) display, an electrophoretic display (EPD), or a light emitting diode (LED) display may be used.

The illumination unit 61 functions as a backlight of the display panel 60. The illumination unit 61 is connected to the front surface 20f as a first surface of the circuit board 20. Since the illumination unit 61 is connected to the circuit board 20 as described above, routing of a wiring for connection can be made the minimum, and it is also possible to block light emitted from the illumination unit 61 with the circuit board 20, and can thus to reduce the influence of stray light on the optical sensor unit 40.

The secondary battery 70 has both of polarity terminals connected to the circuit board 20 via a connection board (not illustrated), and supplies power to a circuit controlling a power source. The power is converted into predetermined voltages by the circuit, so as to be supplied to respective circuits, and thus to drive a circuit which drives the optical sensor unit 40 to measure a pulse, a circuit which drives the display panel 60, and a control circuit (CPU 21) which controls the respective circuits. Charging (power storage) of the secondary battery 70 is performed via a pair of charging terminals which are electrically connected to the circuit board 20 via a conduction member (not illustrated) such as a coil spring, or is charged by using power generated by the solar battery unit 80.

The solar battery (solar cell) unit 80 generates power by converting light energy of external light such as sunlight into power by using a photoelectromotive force effect, and stores power in the secondary battery 70. The solar battery unit 80 of the present embodiment is formed of the single solar panel 82 having the light reception surface 82a. In this configuration, the annular solar battery unit 80 using the single solar panel 82 is exemplified, but the solar battery unit 80 may be formed of a plurality of separate solar panels. In a case where the solar battery unit 80 is formed of a plurality of solar panels, any number of panels may be used. Any shapes of solar panels forming the solar battery unit 80 may be used. The solar battery unit 80 may be formed of a film instead of a solar panel.

A storage unit 180 stores biological information such as a pulse wave from the optical sensor unit 40, position information from the GPS reception unit 160, and body motion information from the body motion sensor unit 170, under the control of the CPU 21.

According to the wrist apparatus 200 as a portable electronic apparatus, in a case where the rear surface 31r of the case main body 31 which is a contact surface of the case section 35 is brought into contact with and mounted on a mounting part of the user, for example, the wrist of the user with the band portion 10, the solar battery unit 80 and the display unit 50 electrically connected to the circuit board 20 disposed in the case section 35 are provided side by side on the front surface 31f as a first surface located on an opposite side to the rear surface 31r as a second surface. As mentioned above, since the solar battery unit 80 and the display unit 50 are disposed side by side on an opposite side to the rear surface 31r, that is, on the front surface 31f, thinning of the case section 35 can be realized more than in a case where the solar battery unit 80 and the display unit 50 are disposed to overlap each other. Since the solar battery unit 80 and the display unit 50 are disposed side by side on the front surface 31f, disposition balance of the apparatus main body 30 can be maintained, and thus it is possible to implement the wrist apparatus 200 as a portable electronic apparatus having favorable mountability while performing power generation in the solar battery unit 80.

According to the wrist apparatus 200, there is provided the optical sensor unit 40 which is disposed in the case section 35 and measures biological information of a user, and the light transmissive portion 44 of the optical sensor unit 40 is exposed from the rear surface 31r of the case main body 31. Therefore, the light transmissive portion 44 can be reliably brought into contact with the user, and thus it is possible to suppress external light from entering the light receiving portion 41. Consequently, it is possible to measure biological information with high accuracy by reducing the influence of external light on the optical sensor unit 40.

As described above, according to the wrist apparatus 200 of the present embodiment, even the small-sized wrist apparatus 200 can secure a large power generation amount so as to make up for power shortage, and thus it is possible to obtain information such as biological information or body motion information in various sensors such as the optical sensor unit 40.

### 4. Modification Examples of Configuration of Wrist Apparatus

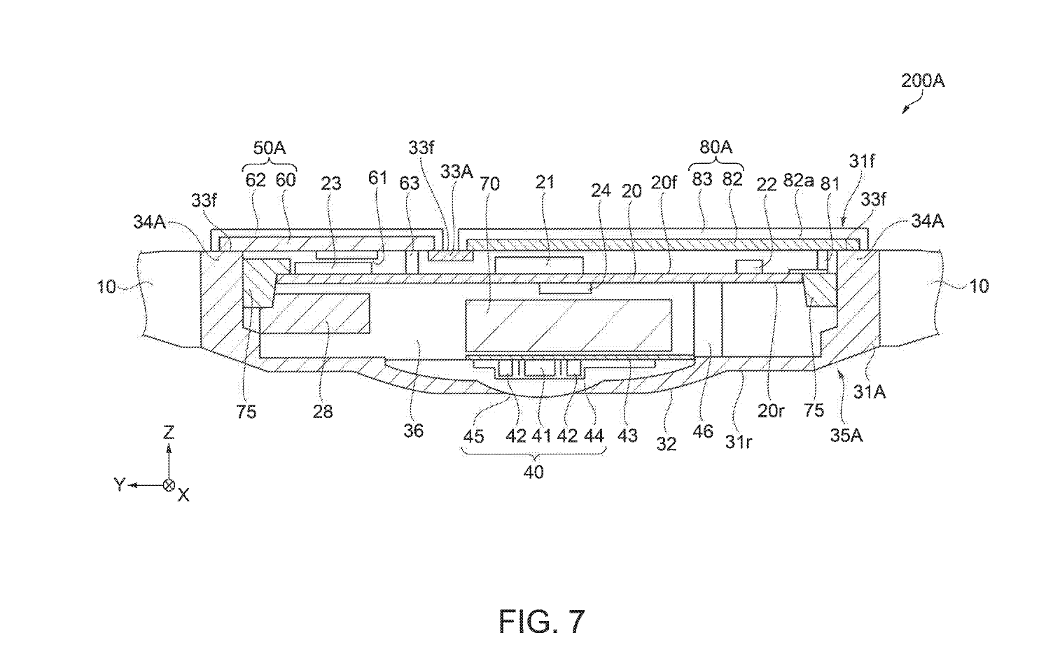

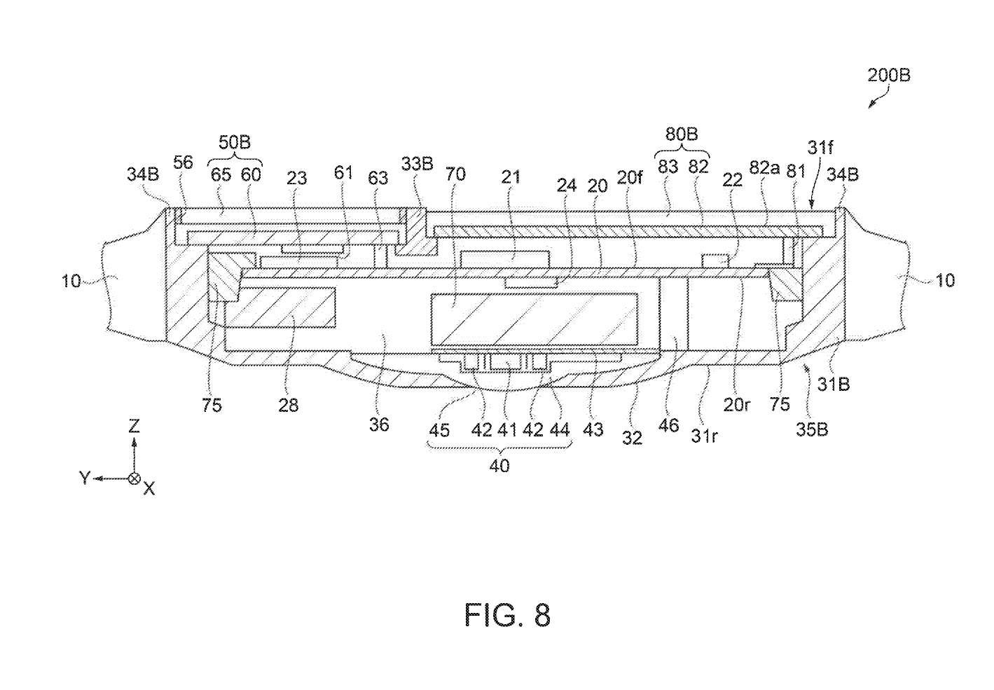

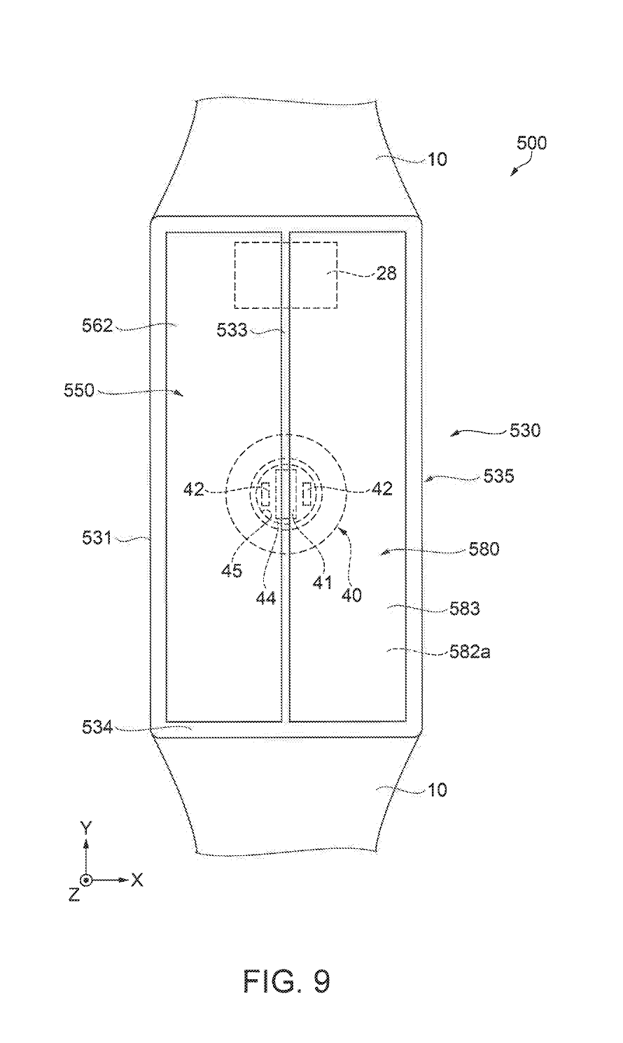

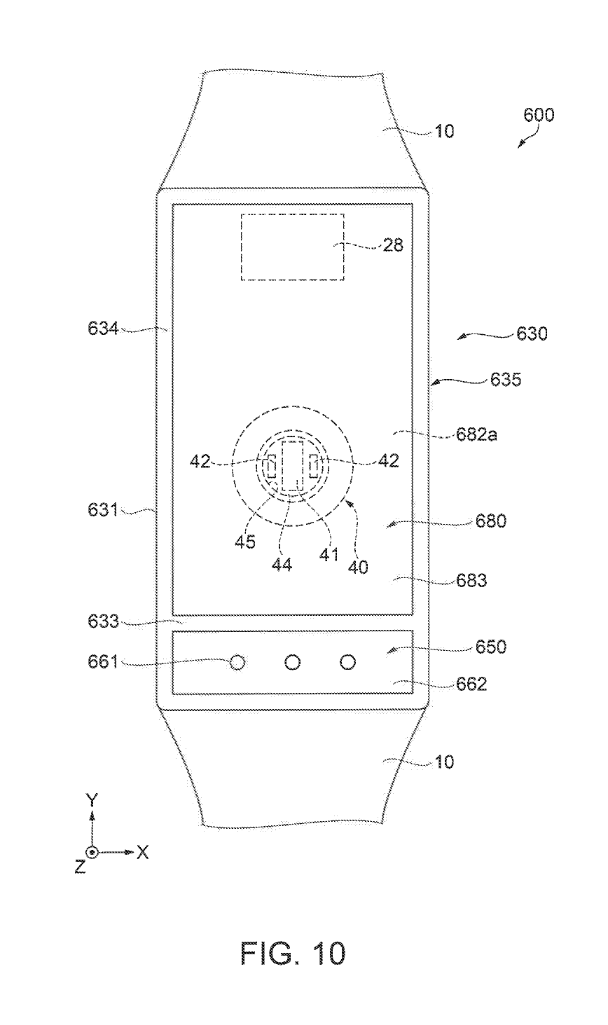

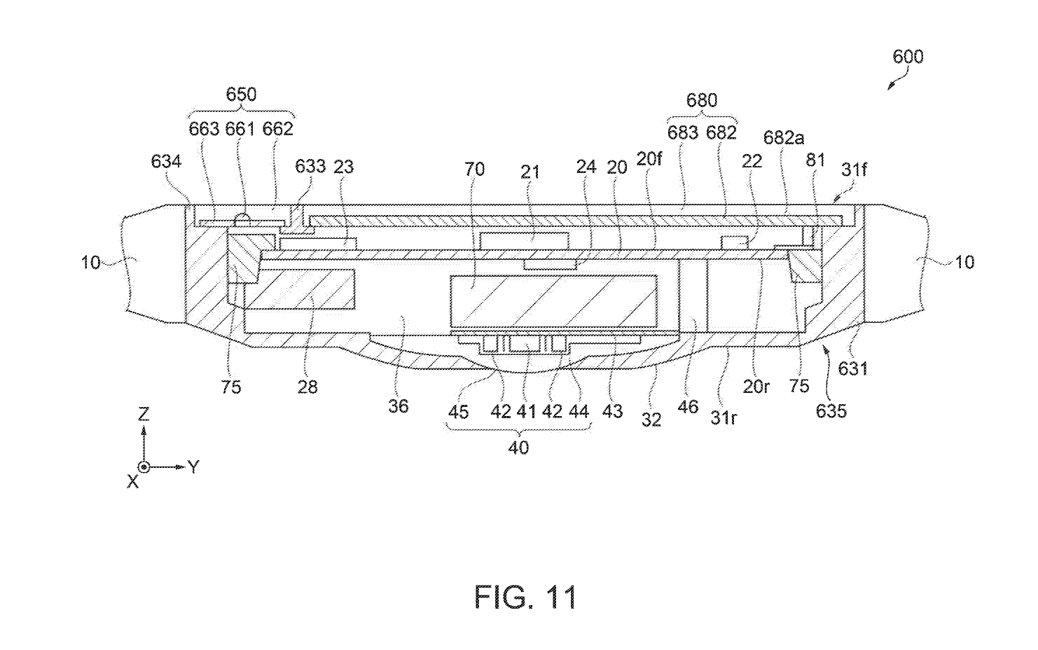

Dispositions or configurations of the solar battery unit 80 and display unit 50 in the wrist apparatus 200 are not limited to the configuration of the above-described embodiment, and may be realized as in modification examples described below. Hereinafter, with reference to FIGS. 7 to 11, Modification Examples 1 to 4 of configurations of the wrist apparatus will be described in this order. Here, FIG. 7 is a sectional view illustrating Modification Example 1 related to a configuration of the wrist apparatus. FIG. 8 is a sectional view illustrating Modification Example 2 related to a configuration of the wrist apparatus. FIG. 9 is a plan view illustrating Modification Example 3 related to a configuration of the wrist apparatus. FIG. 10 is a plan view illustrating Modification Example 4 related to a configuration of the wrist apparatus. FIG. 11 is a sectional view illustrating Modification Example 4 related to a configuration of the wrist apparatus.

### Modification Example 1 of Wrist Apparatus

First, with reference to FIG. 7, a description will be made of Modification Example 1 of a configuration of the wrist apparatus. As illustrated in FIG. 7, a display unit 50A and a solar battery unit 80A are disposed side by side on an opening side (front side) of a case main body 31A in a case section 35A of a wrist apparatus 200A according to Modification Example 1. The case main body 31A which is open on the front side has outer frame portions 34A provided along an outer periphery of the opening, and an intermediate frame portion 33A which connects the opposing outer frame portions 34A to each other, and thus divides an opening region formed by the outer frame portions 34A into two regions.

The display unit 50A is disposed in one region obtained through division using the outer frame portions 34A and the intermediate frame portion 33A, and are mounted on a front surface 33f of the outer frame portions 34A and the intermediate frame portion 33A so as to be attached. The solar battery unit 80A is disposed in the other region obtained through division using the outer frame portions 34A and the intermediate frame portion 33A, and are mounted on a front surface 33f of the outer frame portions 34A and the intermediate frame portion 33A so as to be attached. An internal space 36 which is a closed space is provided in the inside of the case main body 31A surrounded by the case main body 31A, and the display unit 50A and the solar battery unit 80A closing the opening of the case main body 31A.

The display unit 50A includes a display panel 60 mounted on the front surface 33f of the outer frame portions 34A and the intermediate frame portion 33A in one region, and a second protection member 62 which covers the display panel 60 from the front side (+Z axis direction side) and is provided to protect the display panel 60 exposed to the front side. In the display unit 50A with this configuration, the second protection member 62 is exposed to a front surface 31f side as a first surface located on an opposite side to a rear surface 31r as a second surface which is a contact surface of the case main body 31A. In other words, in the display unit 50A located on the front side of the case section 35A, the second protection member 62 is exposed to the front surface 31f side. Therefore, the display unit 50A (display panel 60) located on the front surface 31f side can be protected by the second protection member 62. The second protection member 62 may employ a light-transmissive resin coating material or glass coating material. The display unit 50A may include an illumination unit 61 which irradiates the display panel 60 with light.

The solar battery unit 80A includes the solar panel 82 attached to the front surface 33f of the outer frame portions 34A and the intermediate frame portion 33A in the other region, and a first protection member 83 which covers a front side (+Z axis direction) and a side surface of the light reception surface 82a of the solar panel 82 and is provided to protect the solar panel 82 exposed to the front side. In the solar battery unit 80A with this configuration, the first protection member 83 is exposed to the front surface 31f side as a first surface located on an opposite side to the rear surface 31r which is a contact surface of the case main body 31A. In other words, in the solar battery unit 80A located on the front surface side of the apparatus main body 30A, the first protection member 83 is exposed to the front surface 31f side. Therefore, the solar battery unit 80A (solar panel 82) located on the front side can be protected by the first protection member 83. The first protection member 83 may employ a light-transmissive resin coating material or glass coating material.

An area of the solar panel 82 (an area of the light reception surface 82a) forming the solar battery unit 80A is preferably larger than an area of the display panel 60 (display unit 50A) in a plan view along a direction from the display unit 50A toward the circuit board 20 (a plan view from the +Z axis direction). As mentioned above, since an area of the solar panel 82 (an area of the light reception surface 82a) is larger than an area of the display panel 60 (display unit 50A), a sufficient power generation amount in the solar battery unit 80A for securing power in the wrist apparatus 200A can be obtained.

The rear surface 31r which is a contact surface with a user's body, a protrusion portion 32 protruding from the rear surface 31r, and a measurement window portion 45 of the optical sensor unit 40 which is open to the top of the protrusion portion 32 are provided on the rear side of the case main body 31A. The optical sensor unit 40 as a biological sensor is disposed at a position corresponding to the measurement window portion 45 in a plan view from the +Z axis direction, and a light transmissive portion 44 forming the optical sensor unit 40 is inserted into the measurement window portion 45. Such a configuration is the same as that in the above-described embodiment, and thus a detailed description thereof will be omitted.

In the same manner as in the above-described embodiment, as element components forming the wrist apparatus 200A, for example, a circuit board 20, an azimuth sensor 22 and an acceleration sensor 23, a GPS antenna 28, the optical sensor unit 40, the illumination unit 61 of the display panel 60, and a secondary battery 70 (lithium secondary battery) are stored in the internal space 36 of the case main body 31A. The circuit board 20 is connected to connection wires with the above-described element components, a central processing unit (CPU) 21 which is a control circuit controlling the respective sensors or the display unit 50A or a control circuit including a drive circuit, and other circuit elements 24. Such a configuration is the same as that in the above-described embodiment, and thus a detailed description thereof will be omitted.

According to the wrist apparatus 200A of Modification Example 1, it is possible to achieve the same effect as that in the above-described embodiment. For example, according to the wrist apparatus 200A worn by a user, the solar battery unit 80A and the display unit 50A electrically connected to the circuit board 20 disposed in the case section 35A are provided side by side on the front surface 31f side located on an opposite side to the rear surface 31r. As mentioned above, since the solar battery unit 80A and the display unit 50A are disposed side by side on an opposite side to the rear surface 31r, that is, on the front surface 31f, disposition balance of the case section 35A can be maintained, and thus it is possible to implement the wrist apparatus 200A as a portable electronic apparatus having favorable mountability while performing power generation in the solar battery unit 80A.

### Modification Example 2 of Wrist Apparatus

Next, with reference to FIG. 8, a description will be made of Modification Example 2 of a configuration of the wrist apparatus. As illustrated in FIG. 8, a display unit 50B and a solar battery unit 80B are disposed side by side on an opening side (front side) of a case main body 31B in a case section 35B of a wrist apparatus 200B according to Modification Example 2. The case main body 31B which is open on the front side has outer frame portions 34B provided along an outer periphery of the opening, and an intermediate frame portion 33B which connects the opposing outer frame portions 34B to each other, and thus divides an opening region formed by the outer frame portions 34B into two regions. The outer frame portions 34B and the intermediate frame portion 33B are provided with step parts at inner peripheries on the two separate regions sides.

The display unit 50B is disposed in one region obtained through division using the outer frame portions 34B and the intermediate frame portion 33B, and is inserted into the step parts of the outer frame portions 34B and the intermediate frame portion 33B so as to be attached. The solar battery unit 80B is disposed in the other region obtained through division using the outer frame portions 34B and the intermediate frame portion 33B, and is inserted into the step parts of the outer frame portions 34B and the intermediate frame portion 33B so as to be attached. An internal space 36 which is a closed space is provided in the inside of the case main body 31B surrounded by the case main body 31B, and the display unit 50B and the solar battery unit 80B closing the opening of the case main body 31B.

The display unit 50B includes a liquid crystal display (hereinafter, referred to as a display panel 60) attached to the outer frame portions 34B and the intermediate frame portion 33B in one region, and a windshield plate (in this example, a glass plate) 65 which is located over (+Z axis direction side) the display panel 60, and is attached to the outer frame portions 34B and the intermediate frame portion 33B with a gap with the display panel 60 in one region. The windshield plate (in this example, a glass plate) 65 is a transparent plate which covers the upper side of the display panel 60 and protects the display panel 60 located inward. The windshield plate 65 corresponds to the second protection member 62 described in the embodiment or Modification Example 1, and can protect the display unit 50B (display panel 60) located on the front surface side.

The windshield plate 65 is disposed to close an opening of one region formed by the outer frame portions 34B and the intermediate frame portion 33B in a plan view from the +Z axis direction, and are attached to inner peripheries of the outer frame portions 34B and the intermediate frame portion 33B via a joint member 56 such as a packing or an adhesive. The windshield plate 65 is not limited to a glass plate, and may be a member which is a light-transmissive member through which the display unit 50B can be viewed and is made of materials such as transparent plastic other than glass as long as the materials have the strength sufficient to protect the display panel 60 forming the display unit 50B.

The solar battery unit 80B includes the solar panel 82 attached to the outer frame portions 34B and the intermediate frame portion 33B in the other region, and a first protection member 83 which covers the light reception surface 82a of the solar panel 82 from the front side (+Z axis direction side) and is provided to protect the solar panel 82 from the front side. In the solar battery unit 80B with this configuration, the first protection member 83 is exposed to the front surface 31f side as a first surface located on an opposite side to the rear surface 31r which is a contact surface of the case main body 31B. In other words, in the solar battery unit 80B located on the front surface side of the case section 35B, the first protection member 83 is exposed to the front surface 31f side. Therefore, the solar battery unit 80B (solar panel 82) located on the front surface side can be protected by the first protection member 83. The first protection member 83 may employ a light-transmissive resin coating material or glass coating material. The first protection member 83 may cover surfaces including the light reception surface 82a to the side surface of the solar panel 82.

An area of the solar panel 82 (an area of the light reception surface 82a) forming the solar battery unit 80B is preferably larger than an area of the display panel 60 (display unit 50B) in a plan view along a direction from the display unit 50B toward the circuit board 20 (a plan view from the +Z axis direction). As mentioned above, since an area of the solar panel 82 (an area of the light reception surface 82a) is larger than an area of the display panel 60 (display unit 50B), a sufficient power generation amount in the solar battery unit 80B for securing power in the wrist apparatus 200B can be obtained.

The rear surface 31r which is a contact surface with the user's body, a protrusion portion 32 protruding from the rear surface 31r, and the measurement window portion 45 of the optical sensor unit 40 which is open to the top of the protrusion portion 32 are provided on the rear side of the case main body 31B. The optical sensor unit 40 as a biological sensor is disposed at a position corresponding to the measurement window portion 45 in a plan view from the +Z axis direction, and a light transmissive portion 44 is inserted into the measurement window portion 45 forming the optical sensor unit 40. Such a configuration is the same as that in the above-described embodiment, and thus a detailed description thereof will be omitted.

In the same manner as in the above-described embodiment, as element components forming the wrist apparatus 200B, for example, the circuit board 20, an azimuth sensor 22 and an acceleration sensor 23, a GPS antenna 28, the optical sensor unit 40, the illumination unit 61 of the display panel 60, and a secondary battery 70 (lithium secondary battery) are stored in the internal space 36 of the case main body 31B. The circuit board 20 is connected to connection wires with the above-described element components, a central processing unit (CPU) 21 which is a control circuit controlling the respective sensors or the display unit 50B or a control circuit including a drive circuit, and other circuit elements 24. Such a configuration is the same as that in the above-described embodiment, and thus a detailed description thereof will be omitted.

According to the wrist apparatus 200B of Modification Example 2, it is possible to achieve the same effect as that in the above-described embodiment. For example, according to the wrist apparatus 200B worn by a user, the solar battery unit 80B and the display unit 50B of which the front side is protected by the windshield plate 65, electrically connected to the circuit board 20 disposed in the case section 35B, are provided side by side on the front surface 31f side located on an opposite side to the rear surface 31r. As mentioned above, since the solar battery unit 80B and the display unit 50B are disposed side by side on an opposite side to the rear surface 31r, that is, on the front surface 31f, disposition balance of the case section 35B can be maintained, and thus it is possible to implement the wrist apparatus 200B as a portable electronic apparatus having favorable mountability while performing power generation in the solar battery unit 80B.

In the above Modification Example 2, a description has been made of a configuration in which the windshield plate 65 is used as a second protection member protecting the display unit 50B (display panel 60), but a windshield plate may be used as a first protection member (for example, a glass plate) protecting the solar battery unit 80B (solar panel 82). There may be a configuration in which a single windshield plate may be used as a protection member which covers both of the display unit 50B (display panel 60) and the solar battery unit 80B (solar panel 82), and protects the display unit 50B (display panel 60) and the solar battery unit 80B (solar panel 82).

### Modification Example 3 of Wrist Apparatus

Next, with reference to FIG. 9, a description will be made of Modification Example 3 of a configuration of the wrist apparatus. As illustrated in FIG. 9, a display unit 550 and a solar battery unit 580 are horizontally disposed side by side along the X axis direction on an opening side (front side) of a case main body 531 in a case section 535 of a wrist apparatus 500 according to Modification Example 3. The case main body 531 which is open on the front side has outer frame portions 534 provided along an outer periphery of the opening, and an intermediate frame portion 533 which connects the opposing outer frame portions 534 to each other, and thus divides an opening region formed by the outer frame portions 534 into two regions.

The display unit 550 is disposed in one region in the −X axis direction obtained through division using the outer frame portions 534 and the intermediate frame portion 533, and is attached to the outer frame portions 534 and the intermediate frame portion 533. The solar battery unit 580 is disposed in the other region (+X axis direction) obtained through division using the outer frame portions 534 and the intermediate frame portion 533, and is attached to the outer frame portions 534 and the intermediate frame portion 533.

The display unit 550 includes a display panel (not illustrated), and a second protection member 562 which covers a front side (+Z axis direction) of the display panel and is provided to protect the display panel exposed to the front side. In other words, in the display unit 550 located on the front surface side of the case section 535, the second protection member 562 is exposed to the front side. Therefore, the display unit 550 located on the front surface side can be protected by the second protection member 562. The second protection member 562 may employ a light-transmissive resin coating material or glass coating material. A configuration of the display unit 550 is the same as that of the display unit 50, and thus a detailed description thereof will be omitted.

The solar battery unit 580 includes a solar panel (not illustrated) having a light reception surface 582a, and a first protection member 583 which covers a front side (+Z axis direction side) of the light reception surface 582a of the solar panel exposed to the front side and is provided to protect the light reception surface 582a of the solar panel. In other words, in the solar battery unit 580 located on the front surface side of the apparatus main body 530, the first protection member 583 is exposed to the front side. Therefore, the solar battery unit 580 (the light reception surface 582a of the solar panel) located on the front surface side can be protected by the first protection member 583. The first protection member 583 may employ a light-transmissive resin coating material or glass coating material.

A measurement window portion 45 of the optical sensor unit 40 is provided on the rear side of the case main body 531. The optical sensor unit 40 (light emitting portions 42 and a light receiving portion 41) as a biological sensor is disposed at a position corresponding to the measurement window portion 45 in a plan view from the +Z axis direction, and the light transmissive portion 44 forming the optical sensor unit 40 is inserted into the measurement window portion 45. Such a configuration is the same as that in the above-described embodiment, and thus a detailed description thereof will be omitted.

In the same manner as in the above-described embodiment, for example, the GPS antenna 28 and the like forming the wrist apparatus 500 are stored in an internal space of the case main body 531. Stored constituent components are the same as those in the above-described embodiment, and thus a detailed description thereof will be omitted.

According to the wrist apparatus 500 of Modification Example 3, it is possible to achieve the same effect as that in the above-described embodiment. According to the disposition of Modification Example 3, when the wrist apparatus 500 is mounted on the user's wrist, the +X axis side (three o'clock side) of the case main body 531 is located at the fingertip side of a user and is thus hardly hooked by clothes (sleeve) of the user. In other words, the solar battery unit 580 is hardly hooked by clothes (sleeve) of the user, and it is possible to reduce the time for which sunlight is blocked and thus to perform more efficient power generation.

### Modification Example 4 of Wrist Apparatus

Next, with reference to FIGS. 10 and 11, a description will be made of Modification Example 4 of a configuration of the wrist apparatus. As illustrated in FIGS. 10 and 11, a display unit 650 and a solar battery unit 680 are disposed side by side on an opening side (front side) of a case main body 631 in a case section 635 of a wrist apparatus 600 according to Modification Example 4. The case main body 631 which is open on the front side has outer frame portions 634 provided along an outer periphery of the opening, and an intermediate frame portion 633 which connects the opposing outer frame portions 634 to each other, and thus divides an opening region formed by the outer frame portions 634 into two regions. The intermediate frame portion 633 is provided at a position deviated in the −Y axis direction relative to the center of the case main body 631 in the Y axis direction. In other words, the intermediate frame portion 633 is disposed such that an area of a region located in the +Y axis direction is larger.

The display unit 650 is disposed in one region (a region having a smaller area in the −Y axis direction) obtained through division using the outer frame portions 634 and the intermediate frame portion 633, and is attached to the outer frame portions 634 and the intermediate frame portion 633. The solar battery unit 680 is disposed in the other region (a region having a smaller area in the +Y axis direction) obtained through division using the outer frame portions 634 and the intermediate frame portion 633, and is attached to the outer frame portions 634 and the intermediate frame portion 633. An internal space 36 which is a closed space is provided in the inside of the case main body 631 surrounded by the case main body 631, and the display unit 650 and the solar battery unit 680 closing the opening of the case main body 631.

The display unit 650 includes a display board 663 attached to the outer frame portions 634 and the intermediate frame portion 633 in one region, a single light emitting diode (LED) element or a plurality of LED elements, in this example, three LED elements 661 attached to the display board 663, and a second protection member 662 which covers a front side (+Z axis direction side) of the LED elements 661 and the display board 663 and protects the LED elements 661 exposed to the front side. In the display unit 650 with this configuration, the second protection member 662 is exposed to the front surface 31f side as a first surface located on an opposite side to a rear surface 31r as a second surface which is a contact surface of the case main body 631. In other words, in the display unit 650 located on the front surface side of the case section 635, the second protection member 662 is exposed to the front surface 31f side. Therefore, the display unit 650 (LED elements 661) located on the front surface side can be protected by the second protection member 662. The second protection member 662 may employ a light-transmissive resin coating material or glass coating material. The display unit 650 in this example may deliver information through lighting or blinking of the LEDs.

The solar battery unit 680 includes a solar panel 682 attached onto the outer frame portions 634 and the intermediate frame portion 633 in the other region, and a first protection member 683 which covers a front side (+Z axis direction side) and a side surface of a light reception surface 682a of the solar panel 682 and is provided to protect the solar panel 682 exposed to the front side. In the solar battery unit 680 with this configuration, the first protection member 683 is exposed to the front surface 31f side as a first surface located on an opposite side to the rear surface 31r which is a contact surface of the case main body 631. In other words, in the solar battery unit 680 located on the front surface side of the apparatus main body 630, the first protection member 683 is exposed to the front surface 31f side. Therefore, the solar battery unit 680 (solar panel 682) located on the front surface side can be protected by the first protection member 683. The first protection member 683 may employ a light-transmissive resin coating material or glass coating material.

An area of the solar panel 682 (an area of the light reception surface 682a) forming the solar battery unit 680 is set to be larger than an area of the display unit 650 in a plan view along a direction from the solar panel 682 toward the circuit board 20 (a plan view from the +Z axis direction), and occupies most of the front side of the case main body 631. As mentioned above, since the area of the solar panel 682 (an area of the light reception surface 682a) is large, a sufficient power generation amount in the solar battery unit 680 for securing power in the wrist apparatus 600 can be obtained.

A measurement window portion 45 of the optical sensor unit 40 is provided on the rear side of the case main body 631. The optical sensor unit 40 (light emitting portions 42 and a light receiving portion 41) as a biological sensor is disposed at a position corresponding to the measurement window portion 45 in a plan view from the +Z axis direction, and the light transmissive portion 44 forming the optical sensor unit 40 is inserted into the measurement window portion 45. Such a configuration is the same as that in the above-described embodiment, and thus a detailed description thereof will be omitted.

In the same manner as in the above-described embodiment, as element components forming the wrist apparatus 600, for example, the circuit board 20, an azimuth sensor 22 and an acceleration sensor 23, a GPS antenna 28, the optical sensor unit 40, and a secondary battery 70 (lithium secondary battery) are stored in the internal space 36 of the case main body 631. The circuit board 20 is connected to connection wires with the above-described element components, a central processing unit (CPU) 21 which is a control circuit controlling the respective sensors or blinking of the LED elements 661 of the display unit 650 or a control circuit including a drive circuit, and other circuit elements 24. Such a configuration is the same as that in the above-described embodiment, and thus a detailed description thereof will be omitted.

According to the wrist apparatus 600 of Modification Example 4, it is possible to achieve the same effect as that in the above-described embodiment. As mentioned above, since the area of the solar panel 682 (an area of the light reception surface 682a) can be increased, a sufficient power generation amount in the solar battery unit 680 for securing power in the wrist apparatus 600 can be obtained.

Dispositions or configurations of the solar battery units 80, 80A, and 80B and the display units 50, 50A, and 50B in the wrist apparatuses 200, 200A, 200B, 400, and 600 described in the embodiment or the modification examples are not limited to the dispositions or the configurations in the embodiment or the modification examples, and may employ other dispositions or configurations.

In the embodiment, as an example of a positioning system using a position information satellite, a description has been made of the GPS using the GPS satellite 8 as a position information satellite included in a global navigation satellite system (GNSS), but this is only an example. The global navigation satellite system may include other systems such as Galileo (EU), GLONASS (Russia), or BeiDou (China), or a positioning information satellite transmitting a satellite signal, for example, a stationary satellite or a quasi-zenith satellite such as SBAS. In other words, the wrist apparatus 200 may be configured to acquire any one of date information, time information, position information, and speed information obtained by processing electric waves (radio signals) from position information satellites including satellites other than the GPS satellites 8. Instead of the global navigation satellite system, a regional navigation satellite system (RNSS) may be used.

What is claimed is:

1. A portable electronic apparatus comprising:
a solar battery;
a secondary battery that stores power from the solar battery;
a display that displays information to a user;
a circuit board that is electrically connected to the solar battery, the secondary battery, and the display;
a body motion sensor that is provided on the circuit board and detects body motion of the user;
a biological sensor that measures biological information of the user; and
a case that has a first surface on which the solar battery and the display are provided side by side, and a second surface brought into contact with the user.

2. The portable electronic apparatus according to claim 1,
wherein the biological sensor does not overlap the body motion sensor in a plan view from a normal direction to a light reception surface of the solar battery.

3. The portable electronic apparatus according to claim 1,
wherein the solar battery includes a solar panel, and a first protection cover protecting the solar panel,
wherein the display includes a display panel, and a second protection cover protecting the display panel, and
wherein the first protection cover and the second protection cover are disposed on the first surface.

4. The portable electronic apparatus according to claim 3,
wherein the first protection cover and the second protection cover are a same cover.

5. The portable electronic apparatus according to claim 3,
wherein an area of the solar panel is larger than an area of the display panel in a plan view from a normal direction to a light reception surface of the solar battery.

6. The portable electronic apparatus according to claim 1, further comprising:
an antenna that is electrically connected to the circuit board, and is able to receive a radio signal,
wherein the antenna is disposed between the solar panel and the case in a plan view from a normal direction to a light reception surface of the solar battery.

7. The portable electronic apparatus according to claim 1,
wherein the biological sensor is provided on the second surface.

8. The portable electronic apparatus according to claim 7,
wherein the biological sensor includes a light emitter, a light receiver, and a light transmitter, and
wherein the second surface has an opening, and the light transmitter is disposed in the opening.

9. The portable electronic apparatus according to claim 1,
wherein the secondary battery is disposed between the circuit board and the second surface.

10. The portable electronic apparatus according to claim 6,
wherein the secondary battery is disposed between the circuit board and the biological sensor.

11. The portable electronic apparatus according to claim 1,
wherein the secondary battery is disposed between the solar battery and the biological sensor.

12. A portable electronic apparatus comprising:
a case having a first surface and a second surface opposing each other, the second surface being configured to contact a body part of a user when the portable electronic apparatus is mounted on the user;
a circuit board that is arranged in the case;
a solar battery that is arranged on the first surface of the case and electrically connected to the circuit board;
a secondary battery that is electrically connected to the circuit board and stores power from the solar battery;
a display that is arranged side-by-side with the solar battery on the first surface of the case, is electrically connected to the circuit board and displays information to the user;
a body motion sensor that is provided on the circuit board and detects body motion of the user; and
a biological sensor that measures biological information of the user.
